(19) United States
(12) Patent Application Publication
(10) Pub. No.: US 2026/0232926 A1
(43) Pub. Date: Aug. 13, 2026

(54) MEDICINE CUP STRUCTURE AND ASSEMBLY METHOD THEREOF AND ATOMIZATION DEVICE

(71) Applicant: Sanoya Technology Co., Ltd., Taipei City (TW)

(72) Inventor: Shu-Pin HSIEH, Taipei City (TW)

(21) Appl. No.: 19/533,918

(22) Filed: Feb. 9, 2026

(30) Foreign Application Priority Data
Feb. 10, 2025 (TW) 114104916

(51) Int. Cl. A61M 11/00; A61J 1/20

(52) U.S. Cl. CPC A61M 11/00; A61J 1/2055; A61M 2207/00

(57) ABSTRACT

The present invention relates to a medicine cup structure and assembly method thereof and atomization device, which is used to unseal a BFS (blow-fill-seal) container filled with liquid medicine. The medicine cup structure comprises an assembly member and an effusion chamber. The assembly member comprises a first retaining portion and a first join portion. The first retaining portion forms a cylindrical structure, and the effusion chamber comprises an effusion space, a second retaining portion, and a second join portion. The liquid storage member is a BFS container, wherein the liquid storage member enables filling and clasping inside the cylindrical structure and inside the effusion space. Under the condition that the liquid storage member is not damaged, the liquid storage member cannot be rotated in an axial direction of the liquid storage device as the rotation axis.

### BACKGROUND OF THE INVENTION

### (a) Field of the Invention

The present invention relates to a medicine cup structure and atomization device, and more particularly refers to a medicine cup structure for a blow-fill-seal (BFS) container filled with liquid medicine, and enables concurrent unsealing of a BFS container when assembling the medicine cup structure. Hence, especially with regard to a user with trembling hands, the medicine cup structure is able to provide a BFS container unsealing mechanism that does not require additional alignment. The medicine cup structure is not limited by the size of the BFS container, and can be used with BFS containers of different size and volume, and is especially applicable for use with very small BFS containers, such as those of 1 cc volume or less, with which it is better able to prevent the liquid medicine from flowing out of the medicine cup structure when tilted. Moreover, after finishing use of the medicine cup structure, the user only needs to disassemble and open the medicine cup structure to drop out the BFS container, without having to painstakingly and inconveniently use their fingers to remove the BFS container from the medicine cup structure. Accordingly, the medicine cup structure prevents the fingers from touching residual liquid medicine, The medicine cup structure can also prevent the user from causing the liquid medicine to overflow during the assembly process or when in use due to inadvertently applying excessive pressure to the BFS container. Therefore, using the medicine cup structure of the atomization device enables producing the expected atomization effect.

### (b) Description of the Prior Art

An atomization device is mainly used to atomize liquid into fine droplets, which are then dispersed into the air environment to achieve a specific effect. For example, atomization of perfume into fine droplets are then dispersed into the indoor space, thereby achieving the effect of improving environmental comfort. Other examples include atomizers for indoor fragrance, or atomization of a medicinal liquid medicine to carry out atomization treatment, whereby the liquid medicine is atomized for the patient to inhale, which is then indirectly absorbed into the blood through the lung's air sacs to treat diseases including diseases of the respiratory tract, brain, human organs, immune system, and the eyes; or the medicinal liquid medicine is directly atomized onto the affected area, such as a liquid medicine atomizer used in an Ear, Nose and Throat Clinic.

Regarding existing atomization devices, such as that disclosed in Taiwan patent No. I723765 (hereinafter referred to as Document 1), which comprises a container for storing a liquid (or referred to as a liquid medicine) and a vibrating mechanism for atomizing the liquid medicine. Such an existing atomization device requires the user to pour the liquid medicine into the container before use thereof, thus, the user must first measure the amount of liquid medicine to be used; or if the user wants to replace the liquid medicine with a different liquid medicine, then the user must disassemble and clean the container before reuse, and is thus somewhat inconvenient to use.

In order to improve the problem of the invention described in the aforementioned Document 1. Taiwan patent No. I734450 (hereinafter referred to as Document 2) discloses a medicine cup structure for unsealing a BFS container filled with liquid medicine. According to the disclosure in Document 2, the BFS container is inserted into the medicine cup structure through an opening in a flat-shaped (or referred to as a plate-shaped) opening, then, the front end of the BFS container is rotated relative to the main body of the BFS container to unseal and allow the liquid medicine to enter the medicine cup structure. However, during the operating procedure, the user must hold the main body of the BFS container to enable aligning and inserting the front end of the flat BFS container into the flattened opening of the above-described medicine cup structure. This is not a serious problem for medical staff, but for the elderly or critically ill patients with trembling hands, it is difficult to carry out the aforementioned alignment. In addition, as described in Document 2, force must be applied to the main body of the BFS container to carry out rotation thereof. Because the BFS container is an elastically compressible and flexible plastic object, made from material such as polyethylene (PE) or polypropylene (PP), therefore, in order to unseal the BFS container without difficulty, the user will usually exert excessive force on the main body of the BFS container, resulting in the liquid medicine spilling out, and thus unable to produce the expected atomization effect when using such a medicine cup structure for the atomization device. Document 2 also describes the risk of the liquid medicine flowing out of the medicine cup structure. Because the flattened opening is normally connected to the external environment, thus, when the medicine cup structure is tilted, the liquid medicine will flow out from the flattened opening to the external environment and easily contaminated thereby. Moreover, Document 2 does not disclose what kind of structural configuration and mechanism enables the front end of the BFS container to rotate relative to the main body of the BFS container and enable unsealing thereof. Therefore, the technology disclosed in Document 2 is incomplete and questionable.

### SUMMARY OF THE INVENTION

In light of the above, an object of the present invention lies in providing a medicine cup structure and an atomization device for use therewith, which is used to unseal a BFS (blow-fill-seal) container filled with liquid medicine, which enables concurrent unsealing of the BFS container during assembly of the medicine cup structure, therefore, the medicine cup structure can provide an unsealing mechanism for the BFS container while eliminating the need for additional alignment, which is especially helpful for a user with trembling hands. Furthermore, it's preferred that after unsealing the BFS container, the medicine cup structure assumes a liquid-tight state, which enables preventing the liquid medicine from flowing out of the medicine cup structure when the medicine cup structure is tilted, as well as preventing the liquid medicine from being contaminated by the external environment. The medicine cup structure can also prevent spillage of the liquid medicine caused by the user accidentally applying too much pressure to the main body of the BFS container during the assembly process or when in use. Hence, using the atomization device of the medicine cup structure can produce the expected atomization effect.

Another object of the present invention lies in providing an assembly method for the medicine cup structure.

Based on the above-described objects, the present invention provides a medicine cup structure, which is applicable for use to unseal a blow-fill-seal container (BFS container) filled with liquid medicine, wherein the medicine cup structure comprises: an assembly member, which comprises a first retaining portion and a first join portion. The first join portion is connected to the first retaining portion; the first retaining portion forms a cylindrical structure, and the first join portion is configured to the end portion of the cylindrical structure; and an effusion chamber, which comprises an effusion space, a second retaining portion, and a second join portion, wherein the effusion space is structured from a chamber perimeter wall annular configured to a chamber bottom portion; the chamber bottom portion is connected to the chamber perimeter wall, the second retaining portion is configured inside the effusion space and cannot rotate relative to the chamber perimeter wall and/or the chamber bottom portion. The second join portion is configured to the chamber perimeter wall, wherein the second join portion enables joining to the first join portion, thereby completing assembly of the assembly member and the effusion chamber. The BFS container is a liquid storage member, which can be filled with the liquid medicine and clasped inside the cylindrical structure and the effusion space. Moreover, under the condition of not damaging the liquid storage member, the liquid storage member is impossible to rotate about the axial direction of the liquid storage member as the rotation axis.

Based on the above-described technological characteristics, the assembly member is provided with an assembly member open end, and the first join portion is configured to the assembly member open end. The first join portion is provided with a first threaded structure, the second join portion is provided with a second threaded structure, and the first threaded structure together with the second threaded structure can rotate relative to each other and be fixedly locked.

Based on the above-described technological characteristics, the first join portion is annular configured to the inner side of the cylindrical structure, the first join portion is provided with an internal threaded structure, the second join portion is disposed on the outer surface of the chamber perimeter wall, and is provided with an external threaded structure, and the internal threaded structure together with the external threaded structure can rotate relative to each other and be fixedly locked.

Based on the above-described technological characteristics, the first retaining portion further comprises a first elongated slot structure, and the other end portion of the cylindrical structure is connected to the first elongated slot structure.

Based on the above-described technological characteristics, the second retaining portion comprises a second elongated slot structure.

Based on the above-described technological characteristics, the assembly member is provided with an assembly member closed end corresponding to the assembly member open end. The extremity of the first elongated slot structure is the assembly member closed end, and the assembly member closed end is not connected to the cylindrical structure.

Based on the above-described technological characteristics, the other end portion of the cylindrical structure assumes an arc shape.

Based on the above-described technological characteristics, the second retaining portion is formed by protruding from the chamber perimeter wall and/or the chamber bottom portion, or the second retaining portion is independently formed, and then clasped to the chamber perimeter wall and/or the chamber bottom portion.

Based on the above-described technological characteristics, the assembly member further comprises a window structure, which is formed on the side wall of the cylindrical structure, wherein the window structure is transparent or hollowed out.

Based on the above-described technological characteristics, the effusion chamber is additionally installed with an elastic rubber matrix that covers the surface of the second retaining portion.

Based on the above-described technological characteristics, a microporous membrane is also disposed on the chamber bottom portion, whereby after completing assembly of the assembly member and the effusion chamber, the medicine cup structure assumes a liquid-tight state.

Based on the above-described technological characteristics, the stiffness of the assembly member and/or the effusion chamber is greater than the stiffness of the BFS container.

Based on the above-described technological characteristics, the liquid storage member cannot rotate about the axial direction thereof as the rotation axis, which means that the liquid storage member cannot rotate as a whole body, or the angle that the liquid storage member can be twisted or rotated through is less than 30 degrees.

Based on the above-described objects, the present invention provides an atomization device which comprises the above-described medicine cup structure.

Based on the above-described objects, the present invention further provides an assembly method for the medicine cup structure, which is applicable for use to unseal a blow-fill-seal container (BFS container) filled with liquid medicine, wherein the BFS container is the liquid storage member, which comprises a second assembly portion and a liquid storage portion, wherein the liquid storage portion is connected to the second assembly portion. The liquid storage portion is correspondingly configured with a first end portion and a second end portion; moreover, the extremity of the second end portion is provided with a protruding piece. The liquid storage portion is filled with the liquid medicine, and the protruding piece is connected to the second assembly portion. The assembly method of the medicine cup structure comprises the following steps:
a) Providing an assembly member step: which involves providing the assembly member, wherein the assembly member comprises the first retaining portion and the first join portion, the first join portion is connected to the first retaining portion, the first join portion is provided with a first threaded structure, the first retaining portion comprises the cylindrical structure, the first join portion is configured to the end portion of the cylindrical structure.
b) Providing a liquid storage member step: which involves filling the liquid storage portion of the liquid storage member, and clasping the liquid storage portion inside the cylindrical structure, making it impossible for the liquid storage member to rotate about the axial direction of the liquid storage member as the rotation axis.
c) Providing an effusion chamber step: which involves providing the effusion chamber, comprising the effusion space, the second retaining portion, and the second join portion, wherein the effusion space is structured from the chamber perimeter wall annular configured to the chamber bottom portion, the chamber bottom portion is connected to the chamber perimeter wall, the second retaining portion is configured inside the effusion space, and cannot rotate relative to the chamber perimeter wall and/or the chamber bottom portion, and the second join portion is configured to the chamber perimeter wall, the second join portion is provided with a second threaded structure, and the first threaded structure together with the second threaded structure rotate relative to each other and are fixedly locked.
d) Carrying out a clasping step: which involves rotating the second threaded structure of the second join portion relative to the first threaded structure of the first join portion, thereby enabling the assembly memberand the effusion chamber to gradually approach and tighten together, until the second assembly portion is clasped inside the effusion space; moreover, under the condition of not damaging the liquid storage member, the liquid storage member is unable to rotate around the rotation axis. And,
e) Producing a liquid outlet opening step: which involves continued mutual tightening of the second join portion and the first join portion, further causing the second assembly portion to at least partially separate from the liquid storage portion, producing a liquid outlet opening, at which time, the liquid medicine in the liquid storage portion flows out from the liquid outlet opening and enters the effusion space.

Based on the above-described technological characteristics, wherein the liquid storage member comprises the first assembly portion, and the two ends of the liquid storage portion respectively connect to the first assembly portion and the second assembly portion, the first assembly portion is provided with a first plate structure, the second assembly portion is provided with a second plate structure, and the first retaining portion further comprises the first elongated slot structure; moreover, the other end portion of the cylindrical structure is connected to the first elongated slot structure, wherein providing the assembly member step involves inserting the first plate shape structure of the first assembly portion of the liquid storage member into the first elongated slot structure, thereby restricting the first assembly portion to the interior of the first elongated slot structure, and preventing rotation around the rotation axis.

Based on the above-described technological characteristics, the second retaining portion comprises the second elongated slot structure, wherein carrying out the clasping step enables inserting the second plate structure of the second assembly portion into the second elongated slot structure, thereby restricting the second assembly portion to the interior of the second elongated slot structure, and preventing rotation around the rotation axis.

To enable a further understanding of said objectives, structures, characteristics, and effects, as well as the technology and methods used in the present invention and effects achieved, a brief description of the drawings is provided below followed by a detailed description of the preferred embodiments.

### BRIEF DESCRIPTION OF THE DRAWINGS

FIG. 1 is a schematic view of a first embodiment of an atomization device of the present invention.

FIG. 2 is a partial exploded three-dimensional view of a medicine cup structure separated from a base of the present invention.

FIG. 3 is a three-dimensional view of the medicine cup structure of the present invention.

FIG. 4 is a partial three-dimensional cross-sectional view of an assembly member and an effusion chamber of the medicine cup structure of the present invention.

FIG. 5 is an exploded three-dimensional view of the medicine cup structure of the present invention.

FIG. 6 is a cross-sectional view of the medicine cup structure of the present invention.

FIG. 7 is a three-dimensional schematic view showing relative rotation of a liquid storage portion and a second assembly portion of a liquid storage member of the medicine cup structure of the present invention.

FIG. 8 is a schematic view of a liquid outlet opening formed after the assembly member of the medicine cup structure together with the liquid storage member are rotated relative to the effusion chamber.

FIG. 9 is a schematic view of the medicine cup structure of FIG. 8 mounted on the base enabling a vibration module to atomize the liquid medicine.

FIG. 10 is a three-dimensional view of another embodiment of the medicine cup structure of the present invention.

FIG. 11 is a three-dimensional view of another embodiment of the medicine cup structure of the present invention additionally installed with an elastic rubber matrix.

### DETAILED DESCRIPTION OF THE PREFERRED EMBODIMENTS

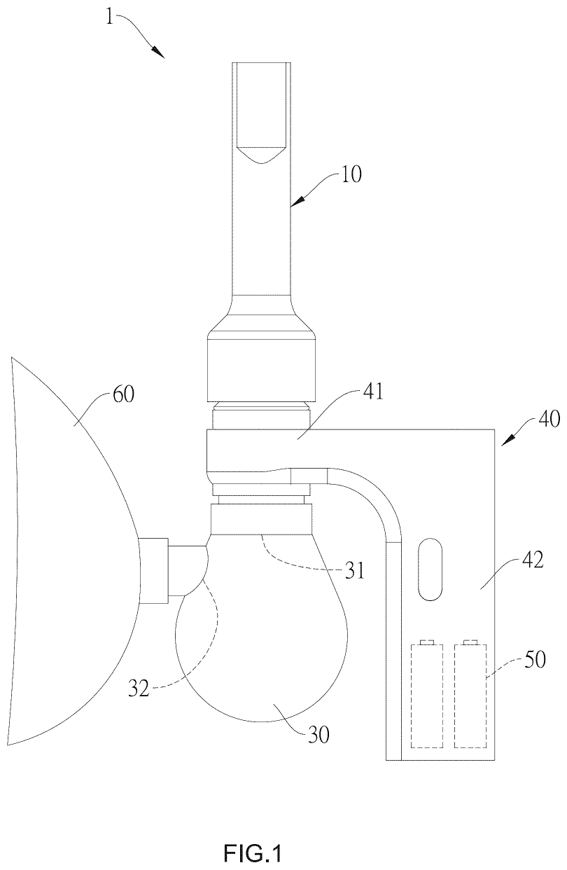

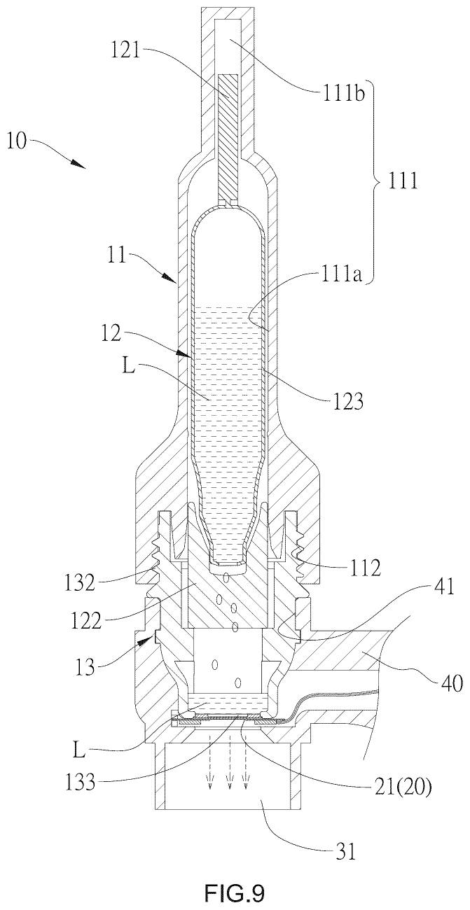

Referring to FIG. 1, which shows an embodiment of an atomization device of the present invention, wherein the embodiment of an atomization device 1 comprises a medicine cup structure 10, a vibration module 20 (please refer in conjunction with FIG. 9), an atomization chamber 30, a base 40, a power supply module 50, and an atomized droplet guide member 60, wherein the atomization device 1 in the embodiment provides an example of a liquid medicine atomizer for medical use but is not limited by such.

The base 40 of the embodiment assumes an L-shaped or linear form, and in the present embodiment assumes an L-shaped form as an example, wherein one end of the base 40 is provided with a connecting channel 41, and the other end is provided with a retaining chamber 42. The two corresponding ends of the connecting channel 41 respectively connect to the medicine cup structure 10 and the atomization chamber 30. The atomization chamber 30 is configured close to the retaining chamber 42 of the base 40, whereas the medicine cup structure 10 is configured at a distance from the retaining chamber 42. The vibration module 20 is fitted inside the connecting channel 41, and the power supply module 50 is installed inside the retaining chamber 42 of the base 40 and is connected to the vibration module 20 through a conducting wire (not shown in the diagrams). A switch installed on an exterior surface of the base 40 enables a user to connect the power supply module 50 to the vibration module 20, causing the power supply module 50 to provide power to the vibration module 20.

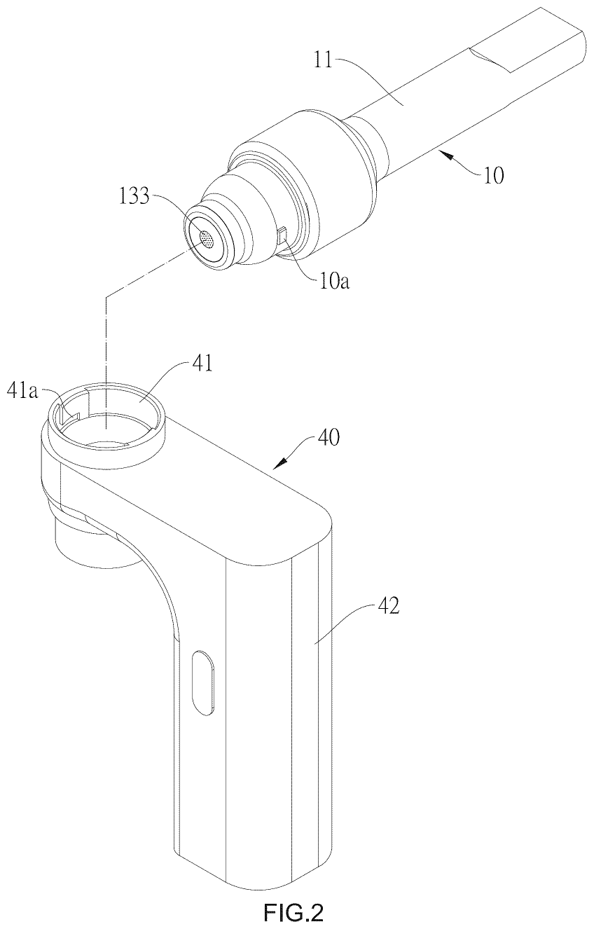

Referring to FIG. 2, which shows how the medicine cup structure 10 is mounted onto an upper square end of the connecting channel 41 of the base 40. The medicine cup structure 10 is also detachable from the connecting channel 41 of the base 40, wherein the medicine cup structure 10 can be designed to be disposable (one-time operation) and thus used to prevent reuse leading to contamination between different medications; whereas the vibration module 20 (as sown in FIG. 9), the base 40, and the power supply module 50 (as sown in FIG. 1) are designed for repeated use to save use-cost. The atomization chamber 30 and the atomized droplet guide member 60 are designed to be disposable or reusable according to requirements. Clasping pieces 10a are provided on the medicine cup structure 10 as a joining method used to join the medicine cup structure 10 to the base 40, and are used to clasp into grooves 41a in the inner wall of the connecting channel 41, thereby enabling the medicine cup structure 10 to be fixedly positioned in the connecting channel 41 of the base 40. For example, rotating the medicine cup structure 10 clockwise enables the clasping pieces 10a to clasp into the grooves 41a, thereby joining the medicine cup structure 10 to the base 40, and the medicine cup structure 10 is rotated in an anticlockwise direction when the medicine cup structure 10 needs to be separated from the base 40, thus facilitating operation for the user.

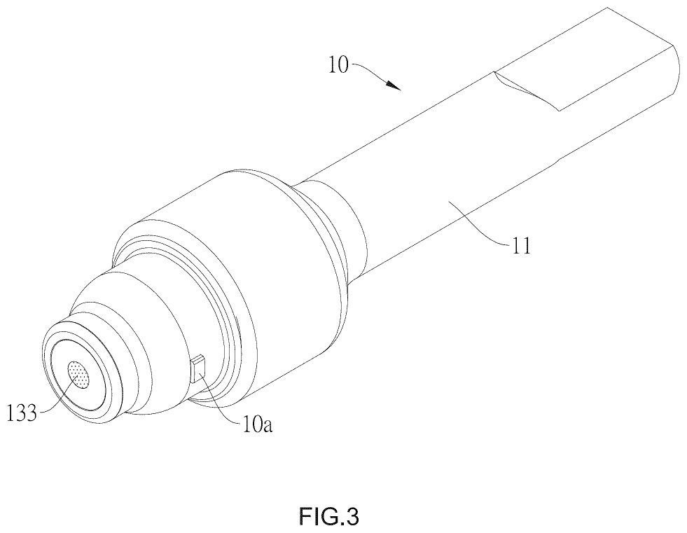

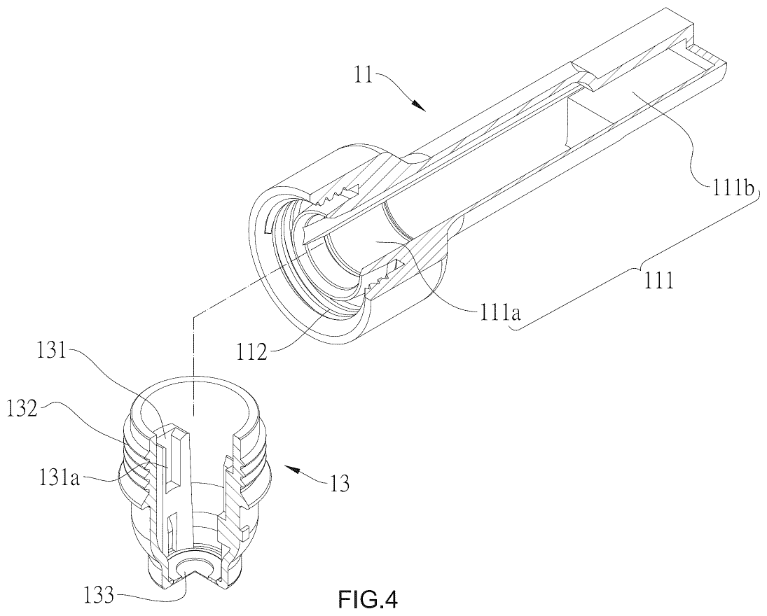

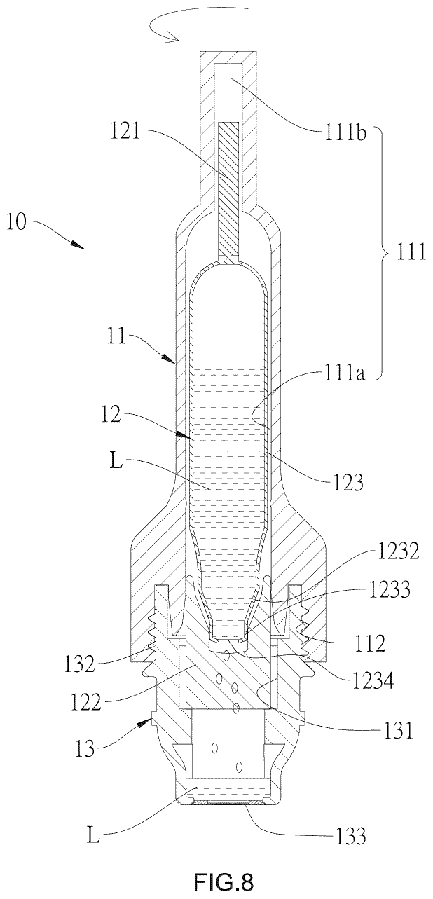

Referring to FIGS. 3, 4 and 5, which show that the medicine cup structure 10 of the present embodiment comprises an assembly member 11 and an effusion chamber 13, wherein the assembly member 11 is detachable mounted on the effusion chamber 13. A liquid storage member 12 is fitted inside the assembly member 11, and extends into the effusion chamber 13, wherein the liquid storage member 12 is a BFS container filled with a liquid medicine L (referring in conjunction with FIGS. 8 and 9).

The BFS container is an elastic compressible and flexible plastic object, such as polyethylene (PE) or polypropylene (PP). Regarding the BFS container, the liquid storage member 12 comprises a first assembly portion 121, a second assembly portion 122, and a liquid storage portion 123, wherein the liquid storage portion 123 is a cylindrical shape, and the two ends thereof respectively connect to the first assembly portion 121 and the second assembly portion 122. A first end portion 1231 and a second end portion 1232 are correspondingly configured on the two ends of the liquid storage portion 123, wherein the first end portion 1231 and the second end portion 1232 are formed as arc-shaped structures. Moreover, the extremity of the second end portion 1232 is provided with a cylindrical shaped protruding piece 1233 having a diameter less than the liquid storage portion 123. The liquid storage portion 123 is filled with the liquid medicine L (referring in conjunction with FIG. 8), and the protruding piece 1233 is connected to the second assembly portion 122. The first assembly portion 121 can be formed with a first plate structure, and the second assembly portion 122 can then be formed with a second plate structure. The first assembly portion 121 is connected to the first end portion 1231, and the first assembly portion 121 is parallel to the axial direction of the cylindrical liquid storage portion 123 (the direction of the cylindrical extended length thereof). The second assembly portion 122 is only connected to the protruding piece 1233 of the second end portion 1232. The structure of the second assembly portion 122 positioned on two sides of the protruding piece 1233 is symmetrical, and the second end portion 1232 assumes a separated state. Accordingly, after the second assembly portion 122 has been fixedly retained and rotated about the axial direction of the liquid storage portion 123 as the rotation axis, the protruding piece 1233 separates from the second assembly portion 122, producing a liquid outlet opening 1234 (see FIG. 8). It is understood that in a design of another embodiment of the present invention, the protruding piece 1233 separating from the second end portion 1232 produces the liquid outlet opening 1234, or in a design of another embodiment of the present invention, breaking of the protruding piece 1233 or the second end portion 1232 produces the liquid outlet opening 1234. In other words, after the second assembly portion 122 has been fixedly retained and rotated about the axial direction of the liquid storage portion 123 as the rotation axis, at least partial breakage of the liquid storage portion 123 produces the liquid outlet opening 1234. For example, at least partial separation of the second assembly portion 122 from the liquid storage portion 123 and the protruding piece 1233 or the second end portion 1232 produces the liquid outlet opening 1234. It is hereby specifically pointed out that in the embodiment where the liquid storage member 12 is structurally symmetrical, the axial axis of rotation of the liquid storage portion 123 is the same as that of the liquid storage member 12, because a large proportion of BFS containers are designed with symmetrical structures, thus, for the sake of brevity, the axial direction or rotational axis described hereinafter can refer to the axial axis of rotation of the liquid storage portion 123 and/or the liquid storage member 12.

The assembly member 11 comprises a first retaining portion 111 and a first join portion 112, wherein the first retaining portion 111 is a hollow tube body; moreover, the assembly member 11 is provided with an assembly member open end 11a, and more preferably, the assembly member 11 is further provided with an assembly member closed end 11b corresponding to the assembly member open end 11a, wherein the first join portion 112 is connected to the first retaining portion 111. For example, the first join portion 112 is configured to the first retaining portion 111, and the first retaining portion 111 comprises a cylindrical structure 111a and a first elongated slot structure 111b. The first join portion 112 is configured to an end portion of the cylindrical structure 111a (forming the assembly member open end 11a), and the other end portion of the cylindrical structure 111a assumes an arc shape which is connected to the first elongated slot structure 111b. The other end portion of the cylindrical structure 111a assuming an arc shape facilitates guiding the first assembly portion 121 into the first elongated slot structure 111b, thereby enabling restricting the first assembly portion 121 to the interior of the first elongated slot structure 111b, and thus making it impossible to rotate about the axial direction of the liquid storage portion 123 as the rotation axis. The cylindrical structure 111a and the first elongated slot structure 111b together form a hollow tube body, and preferably, the extremity of the first elongated slot structure 111b is the assembly member closed end 11b, with the assembly member closed end 11b not connected to the cylindrical structure 111a. In addition, it is preferable that the first join portion 112 is configured to the inner side of the cylindrical structure 111a, and more preferably the first join portion 112 is annular configured to the inner side of the cylindrical structure 111a. Taking the first join portion 112 as having a threaded structure as an example, accordingly, the first join portion 112 in the present embodiment is provided with an internal screw thread structure, it is preferable that the first retaining portion 111 and the first join portion 112 are formed as an integral body, which is formed, for example, through an injection molding manufacturing process; in other words, the assembly member 11 is formed as an integral body. It is preferable that the stiffness of the first retaining portion 111 and/or the first join portion 112 is greater than the stiffness of the liquid storage portion 123. Hence, the first retaining portion 111 and/or the first join portion 112 is/are even more resistant to deformation compared to the liquid storage portion 123. In other words, when an external force or pressure is applied, such as when multiple fingers are used together to apply a holding force, the first retaining portion 111 and/or the first join portion 112 is/are even more able to maintain its/their shape and structure compared to the liquid storage portion 123. It is preferable that the material used to produce the first retaining portion 111 and/or first join portion 112 is either polystyrene, polycarbonate, acrylic, polyethylene terephthalate, or high-density polypropylene.

The effusion chamber 13 is a hollow chamber, comprising an effusion space 130, a second retaining portion 131, and a second join portion 132, wherein the effusion space 130 is structured from a chamber perimeter wall 130a annular configured to a chamber bottom portion 130b. The chamber bottom portion 130b is connected to the chamber perimeter wall 130a, and forms a chamber closed end 13b with a microporous membrane 133 described below. A chamber open end 13a is located at the chamber perimeter wall 130a and configured corresponding to the chamber closed end 13b. The second retaining portion 131 is configured inside the effusion space 130. For example, the second retaining portion 131 is formed by the chamber perimeter wall 130a and/or the chamber bottom portion 130b, and cannot rotate relative to the chamber perimeter wall 130a and/or the chamber bottom portion 130b. In another embodiment, the second retaining portion 131 is separately molded and then clasped to the chamber perimeter wall 130a and/or the chamber bottom portion 130b, thereby preventing it from rotating relative to the chamber perimeter wall 130a and/or the chamber bottom portion 130b.

The second retaining portion 131 comprises a second elongated slot structure 131a, and in subsequent operations the second assembly portion 122 is inserted into the second elongated slot structure 131a, thereby clasping and restricting the second assembly portion 122 inside the second elongated slot structure 131a, and thus making it impossible to rotate about the axial direction of the liquid storage portion 123 as the rotation axis. The second join portion 132 is configured to the chamber perimeter wall 130a, and is provided with a threaded structure. In the present embodiment, the second join portion 132 is configured to the outer surface of the chamber perimeter wall 130a and is provided with a threaded structure, and in subsequent operations the second join portion 132 is joined to the first join portion 112, using a permanent or temporary joining method, for example, such as clasping, engaging, or threading, but is not limited by such. In the present embodiment, the second join portion 132 and the first join portion 112 are respectively provided with a threaded structure, and can be connected to each other by means of their corresponding threaded structures, enabling mutual rotation and fixedly locking through their corresponding threaded structures, thereby enabling the assembly member 11 and the effusion chamber 13 to gradually approach and tighten together, whereby the gradual approach facilitates inserting the second assembly portion 122 into the second elongated slot structure 131a. And after the second assembly portion 122 has been inserted into the second elongated slot structure 131a, whereby, because the second assembly portion 122 is restricted inside the second elongated slot structure 131a, the second assembly portion 122 cannot rotate about the axial direction of the liquid storage portion 123 as the rotation axis. Hence, the continued tightening caused by the mutual corresponding rotation of the second join portion 132 and the first join portion 112 enables the second assembly portion 122 to separate from the protruding piece 1233 to form the liquid outlet opening 1234 (see FIG. 8). In the present embodiment, the second join portion 132 is an external threaded structure and the first join portion 112 is an internal threaded structure. The chamber bottom portion 130b is also fitted with the microporous membrane 133, and because the characteristics, material, effectiveness, and functional mechanism of the microporous membrane 133 are already disclosed in Document 1 and Document 2, thus not further detailed herein. In general, the microporous membrane 133 and the chamber bottom portion 130b are made from different material. It is preferable that the chamber perimeter wall 130a, the chamber bottom portion 130b, the second retaining portion 131, and the second join portion 132 are formed as an integral body, which is formed, for example, through an injection molding manufacturing process. In other words, apart from the microporous membrane 133, the effusion chamber 13 is formed as a single body. It is preferable that the stiffness of the chamber perimeter wall 130a, the chamber bottom portion 130b, the second retaining portion 131, and/or the second join portion 132 is greater than the stiffness of the liquid storage portion 123. It is preferable that the material of the chamber perimeter wall 130a, the chamber bottom portion 130b, the second retaining portion 131, and/or the second join portion 132 is either polystyrene, polycarbonate, acryl resin, polyethylene terephthalate, or high-density polypropylene. It is hereby specifically pointed out that the assembly member 11 and the effusion chamber 13 can be of the same or different material; however, it is preferable that the assembly member 11 and the effusion chamber 13 are of the same material, to save on the types of material preparation and costs during the production and manufacturing process.

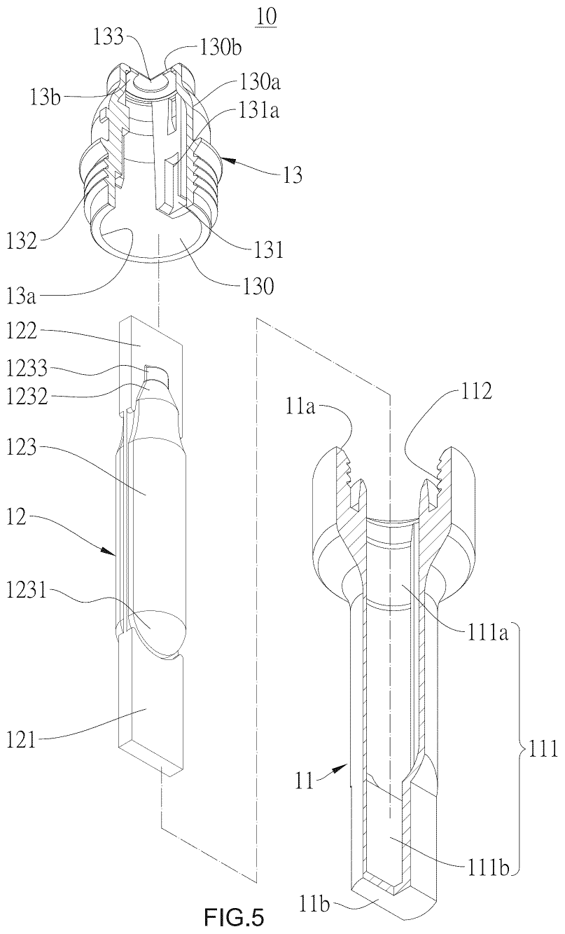

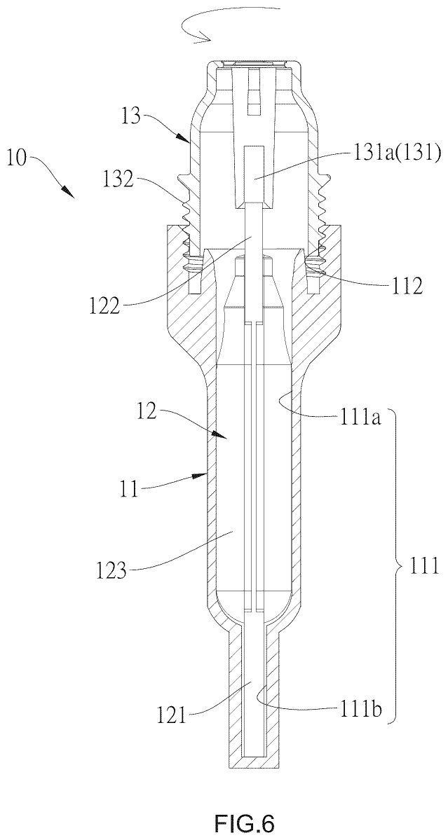

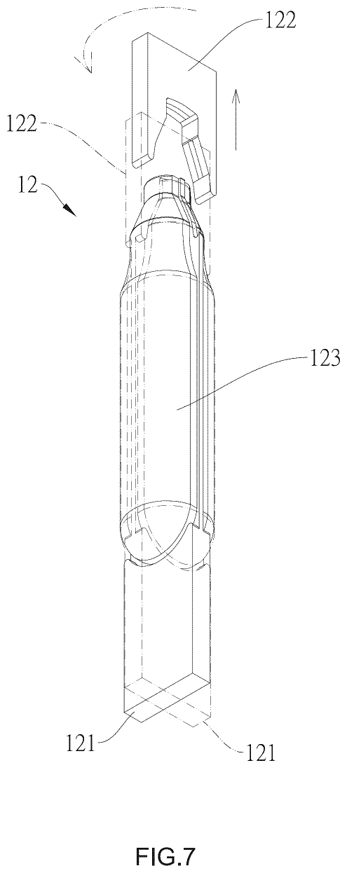

Referring to FIGS. 5, 6, 7 and 8, when assembling the medicine cup structure 10, the assembly method of the medicine cup structure 10 is as follows: first insert the first assembly portion 121 (a first plate shape structure) of the liquid storage member 12 into the cylindrical structure 111a through the assembly member open end 11a, further inserting it into the first elongated slot structure 111b through the cylindrical structure 111a, thereby retaining the liquid storage portion 123 inside the cylindrical structure 111a. As described above, because the other end portion of the cylindrical structure 111a assumes an arc shape, it thus facilitates guiding the first assembly portion 121 into the first elongated slot structure 111b, and eliminates the need for alignment, and therefore enormously helpful for a user with trembling hands. In addition, it is hereby specifically pointed out that even if the other end portion of the cylindrical structure 111a does not assume an arc shape (for example, assumes a flat shape), it can still be used during subsequent assembly whereby the effusion chamber 13 is used to drive rotation of the liquid storage member 12, and enable the first assembly portion 121 to enter the first elongated slot structure 111b, and thus still eliminating the need for alignment, and achieving an enormously helpful function for a user with trembling hands. Furthermore, at this time, the first assembly portion 121 has already been restricted to within the first elongated slot structure 111b and thus cannot rotate about the axial direction of the liquid storage portion 123 as the rotation axis; in other words, the entire liquid storage member 12 (BFS container) has already been restricted to within the first elongated slot structure 111b, and thus cannot rotate about the axial direction of the liquid storage portion 123 as the rotation axis. Next, the external threaded structure of the second join portion 132 is rotated together with the internal threaded structure of the first join portion 112, causing the assembly member 11 and the effusion chamber 13 to gradually approach and tighten together, until the second assembly portion 122 is inserted into the second elongated slot structure 131a. As described above, at which time, because the second assembly portion 122 is already inserted into and restricted inside the second elongated slot structure 131a, it thus cannot rotate about the axial direction of the liquid storage portion 123 as the rotation axis. Next, the continued tightening caused by rotating the second join portion 132 together with the first join portion 112, that is, the continued tightening caused by the rotation of the assembly member 11 together with the effusion chamber 13, thus enables the second assembly portion 122 to separate from the protruding piece 1233 and form the liquid outlet opening 1234. At which time, the liquid medicine L in the liquid storage portion 123 flows out from the liquid outlet opening 1234 and enters the effusion space 130 inside the effusion chamber 13. Based on the design of the microporous membrane 133 disclosed in Document 1 and Document 2, the vibration module 20, to be described later, has not yet been activated, and under the condition whereby a vibrating member 21 has not yet been activated, the liquid medicine L will not pass through the microporous membrane 133 and flow out to the external environment or the atomization chamber 30, to be described later.

Accordingly, during assembly of the medicine cup structure 10, the liquid storage member 12 (BFS container) can be unsealed at the same time, therefore, especially for a user with trembling hands, the medicine cup structure 10 can provide an unsealing mechanism for the liquid storage member 12 (BFS container), eliminating the need for additional alignment. In addition, during and after the unsealing process of the liquid storage member 12, the user holds the assembly member 11 and the effusion chamber 13 with their hands and rotates them relative to each other, without contacting the liquid storage member 12 (BFS container). Furthermore, because the stiffness of both the assembly member 11 and the effusion chamber 13 is greater than the stiffness of the liquid storage member 12, thus, the entire medicine cup structure 10 can also prevent the liquid medicine from overflowing if the user inadvertently applies excessive pressure to the main body (the liquid storage portion 123) of the liquid storage member 12 (BFS container) during the assembly process or when in use, thereby enabling the atomization device 1 of the medicine cup structure 10 to produce the expected atomization effect. Referring to FIG. 9, which shows the liquid medicine L entering the effusion chamber 13, accumulating on top of the microporous membrane 133 located at the bottom portion of the effusion chamber 13. Next, the vibrating member 21 of the vibration module 20 vibrates causing the liquid medicine L to atomize into a plurality of droplets, wherein the atomization chamber 30 comprises a droplet inlet 31 and a droplet outlet 32 (see FIG. 1). The droplets enter the atomization chamber 30 through the droplet inlet 31 and leave the atomization chamber 30 through the droplet outlet 32. Referring to FIG. 1, which shows the atomized droplet guide member 60 provided with a passage to the droplet outlet 32 of the atomization chamber 30, wherein the droplets enter the atomized droplet guide member 60 from the atomization chamber 30, and are guided by the atomized droplet guide member 60 to diffuse in a fixed direction.

It is hereby specifically pointed out that in the present embodiment, when the assembly member 11, the liquid storage member 12, and the effusion chamber 13 are completely assembled, as shown in FIG. 8, that is, the liquid storage member 12 is loaded in the cylindrical structure 111a and inside the effusion space 130, the entire medicine cup structure 10 assumes a liquid-tight state, that is, at this point, the liquid medicine L in the medicine cup structure 10 is in a static state, and will not flow out of the medicine cup structure 10 due to the influence of gravity. Therefore, the liquid medicine L is prevented from flowing out of the medicine cup structure 10 when the medicine cup structure 10 is tilted, thereby preventing the liquid medicine L from being contaminated by the external environment.

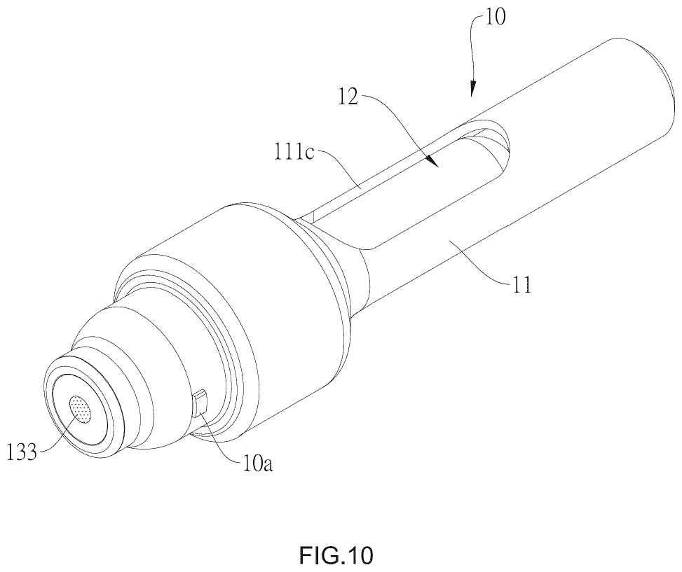

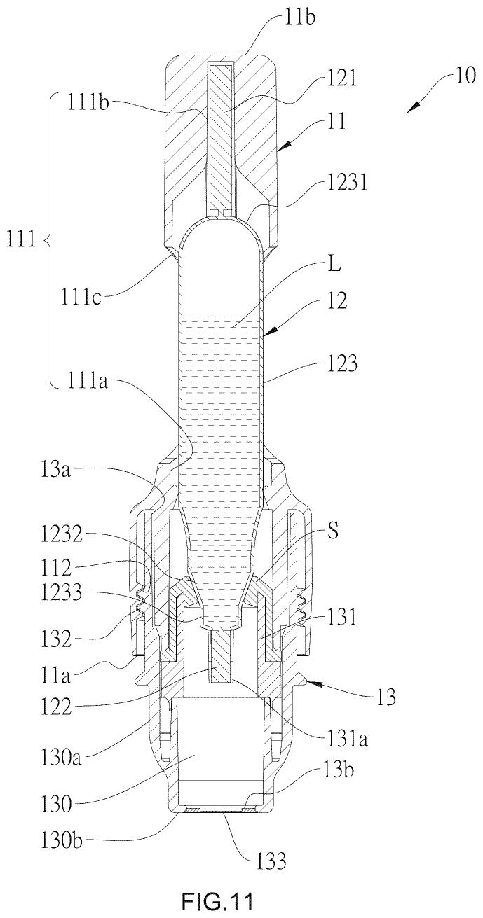

Referring to FIGS. 10 and 11, in another embodiment, the assembly member 11 further comprises a window structure 111c, which is formed on the side wall of the cylindrical structure 111a, wherein the window structure 11c exposes the liquid storage portion 123, enabling the user to view and identify what's inside thereof. The window structure 111c can be a transparent window, such as transparent acrylic or glass, ensuring the entire medicine cup structure 10 maintains a liquid-tight state, or with cost-saving considerations in mind, the window structure 111c can be hollowed out, however, using such a method may inhibit the ability of the medicine cup structure 10 to assume a liquid-tight state, and thus lead to the problem of too high a dissipation rate for the liquid medicine L. And an excessive dissipation rate will result in an insufficient amount of liquid medicine L being atomized, further resulting in a poor effective treatment. In another embodiment of the present invention, see FIG. 11, to solve the problem of an excessive dissipation rate, the effusion chamber 13 is additionally installed with an elastic rubber matrix S that covers the surface of the second retaining portion 131, enabling the elastic rubber matrix S to brace at least a part of the periphery of the second end portion 1232 of the liquid storage portion 123, and thus further able to hold the liquid medicine L in the effusion space 130, thereby solving the problem of an excessive dissipation rate. It is preferable that the elastic rubber matrix S is able to brace the entire 360-degree circumference of the second end portion 1232 of the liquid storage portion 123, and thus able to air-tight seal the liquid medicine L in the effusion space 130, thereby solving the problem of an excessive dissipation rate, wherein the material of the elastic rubber matrix S can be silicone, for example, a silicone membrane or a silicone O-ring.

Various types of the liquid storage member 12 (BFS container) actually come in many different shapes, thus, based on the above-described detailed explanation, the main mechanism of the medicine cup structure 10 of the present invention can be understood as follows: first, under the condition of not damaging the liquid storage member 12, the liquid storage member 12 is clasped inside the cylindrical structure 111a of the assembly member 11 and the effusion space 130 of the effusion chamber 13, and thus making it impossible to rotate about the axial direction of the liquid storage portion 123 as the rotation axis. Next, the liquid storage member 12 is broken, that is, the user further forces continued rotating and tightening of the assembly member 11 together with the effusion chamber 13, thus further causing the second assembly portion 122 to at least partially separate from the liquid storage portion 123 and the protruding piece 1233 or the second end portion 1232 to create the liquid outlet opening 1234. In another embodiment, the first retaining portion 111 comprises the cylindrical structure 111a without the need to configure the first elongated slot structure 111b, whereby the cylindrical structure 111a only needs to prevent the liquid storage member 12 from rotating in the cylindrical structure 111a. For example, under the condition of not damaging the liquid storage member 12, the liquid storage member 12 cannot rotate about the axial direction thereof as the rotation axis. As a specific example, the liquid storage portion 123 of the liquid storage member 12 is a rectangular body, and the cylindrical structure 111a is also a rectangular body; moreover, the hollow tube body of the cylindrical structure 111a is only slightly larger than the liquid storage portion 123, thus, the liquid storage member 12 is unable to rotate in the cylindrical structure 111a. Another example, the cross-section of the liquid storage portion 123 of the liquid storage member 12 is oval-shaped, and the cross section of the hollow tube body of the cylindrical structure 111a is oval-shaped, and is slightly greater than the cross section of the liquid storage portion 123, therefore, the liquid storage member 12 is unable to rotate in the cylindrical structure 111a. Another example, a limiting member is fitted inside the hollow tube body of the cylindrical structure 111a, wherein the limiting member can limit the second assembly portion 122 making it impossible for it to rotate about the axial direction of the liquid storage member 12 as the rotation axis.

In another embodiment, the second retaining portion 131 only needs to prevent the liquid storage member 12 from rotating in the effusion chamber 13. For example, under the condition of not damaging the liquid storage member 12, the liquid storage member 12 cannot rotate about the axial direction thereof as the rotation axis. Apart from the second retaining portion 131 in the above-described embodiment comprising the second elongated slot structure 131a, as an example, other specific examples can include an example such as the second retaining portion 131 configured as a limiting member, which can limit the second assembly portion 122 making it impossible to rotate about the axial direction of the liquid storage member 12 as the rotation axis. For example, the limiting member can be a holding member to hold the second assembly portion 122, making it impossible to rotate about the axial direction of the liquid storage member 12 as the rotation axis. Another example includes the limiting member being a blocking body, which blocks the second assembly portion 122 making it impossible to rotate about the axial direction of the liquid storage member 12 as the rotation axis.

Accordingly, based on the above-described detailed explanation, the assembly method of a medicine cup structure 10 of the present invention can be understood as the assembly method of the medicine cup structure 10 applicable for unsealing a blow-fill-seal container (BFS container) filled with the liquid medicine L, wherein the BFS container is the liquid storage member 12, which comprises the second assembly portion 122 and the liquid storage portion 123, wherein the liquid storage portion 123 is connected to the second assembly portion 122; the first end portion 1231 and the second end portion 1232 are correspondingly configured on the two ends of the liquid storage portion 123; moreover the, the extremity of the second end portion 1232 is provided with the protruding piece 1233. The liquid storage portion 123 is filled with the liquid medicine L, and the protruding piece 1233 is connected to the second assembly portion 122. The assembly method of the medicine cup structure 10 comprises the following steps of:
a) Providing an assembly member step: which involves providing the assembly member 11, comprising the first retaining portion 111 and the first join portion 112; the first join portion 112 is connected to the first retaining portion 111, and the first join portion 112 is provided with a first threaded structure (as the internal threaded structure shown in FIG. 5); the first retaining portion 111 comprises the cylindrical structure 111a, and the first join portion 112 is configured to the end portion of the cylindrical structure 111a.
b) Providing a liquid storage member step: which involves providing the liquid storage member 12, wherein the liquid storage portion 123 of the liquid storage member 12 is filled and clasped inside the cylindrical structure 111a, making it impossible for the liquid storage member 12 to rotate about the axial direction of the liquid storage member 12 as the rotation axis.
c) Providing an effusion chamber step: which involves providing the effusion chamber 13, comprising the effusion space 130, the second retaining portion 131, and the second join portion 132, wherein the effusion space 130 is structured from the annular configured chamber perimeter wall 130a and the chamber bottom portion 130b; the chamber bottom portion 130b is connected to the chamber perimeter wall 130a, the second retaining portion 131 is configured inside the effusion space 130 and cannot rotate relative to the chamber perimeter wall 130a and/or the chamber bottom portion 130b; the second join portion 132 is configured to the chamber perimeter wall 130a, and the second join portion 132 is provided with a second threaded structure (as the external threaded structure shown in FIG. 5), and the first threaded structure together with the second threaded structure can rotate relative to each other and be fixedly locked together.
d) Carrying out a clasping step: which involves rotating the second threaded structure of the second join portion 132 relative to the first threaded structure of the first join portion 112, thereby enabling the assembly member 11 and the effusion chamber 13 to gradually approach each other and tighten together, until the second assembly portion 122 is clasped inside the effusion space 130; moreover, under the condition of not damaging the liquid storage member 12, the liquid storage member 12 is unable to rotate around the rotation axis.
e) Producing a liquid outlet opening step: which involves continued mutual tightening of the second join portion 132 and the first join portion 112, further causing the second assembly portion 122 to at least partially separate from the liquid storage portion 123, producing the liquid outlet opening 1234, at which point, the liquid medicine L in the liquid storage portion 123 flows out from the liquid outlet opening 1234 and enters the effusion space 130.

It is hereby specifically pointed out that the above-described steps are not necessarily in the above-mentioned sequential relationship, the user can adjust the order in which the steps are carried out according to their actual usage situation. In addition, as for the above description, regarding the liquid storage member 12 being unable to rotate in the axial direction thereof (the liquid storage member 12) as the rotation axis, or being impossible for it to rotate about the axial direction of the liquid storage member 12 as the rotation axis, or impossible to rotate about the axial direction of the liquid storage portion 123 as the rotation axis, refers to under the condition of not damaging the liquid storage member 12, it is unable to rotate as a whole or the angle it can be twisted or rotated through is less than 30 degrees.

In conclusion, the medicine cup structure of the present invention certainly achieves the effect to unseal a BFS (blow-fill-seal) container filled with liquid medicine, and enables concurrent unsealing of a BFS container during assembly of the medicine cup structure. Hence, especially for a user with trembling hands, the medicine cup structure can provide an unsealing mechanism for the BFS container that eliminates the need for additional alignment. Furthermore, after unsealing the BFS container, the improved medicine cup structure assumes a liquid-tight state, and thus able to prevent liquid medicine from flowing out of the medicine cup structure when the medicine cup structure is tilted, thus avoiding the liquid medicine from being contaminated by the external environment. The medicine cup structure can also prevent spillage of the liquid medicine during the assembly process or when in use caused by the user accidentally applying too much pressure to the main body of the BFS container. Hence, using the atomization device of the medicine cup structure can produce the expected atomization effect. In addition, the medicine cup structure can further comprise a window structure, which exposes the main body of the BFS container, enabling the user to view and identify what's inside thereof.

It is of course to be understood that the embodiments described herein are merely illustrative of the principles of the invention and that a wide variety of modifications thereto may be effected by persons skilled in the art without departing from the spirit and scope of the invention as set forth in the following claims.

In addition, it is not necessary to achieve all the objects, advantages, or features disclosed in any of the embodiments or claims of the present invention. Further, the abstract section and title are only used to assist in patent document searches, and are intended to limit the claims of the present invention. Further, terms such as “first” and “second” mentioned in this specification or the claims are used only to name elements or operational steps or to distinguish different embodiments or ranges, they are not used to limit the upper or lower limit on the number of components, nor are they used to limit the order in which the operational steps should be carried out.

What is claimed is:

1. A medicine cup structure, which is applicable for use to unseal a blow-fill-seal container (BFS container) filled with liquid medicine, wherein a medicine cup structure comprises:
an assembly member, which comprises a first retaining portion and a first join portion, the first join portion is connected to the first retaining portion, the first retaining portion comprises a cylindrical structure, the first join portion is configured to an end portion of the cylindrical structure; and
an effusion chamber, which comprises an effusion space, a second retaining portion, and a second join portion, wherein the effusion space is structured from a chamber perimeter wall annular configured to a chamber bottom portion, the chamber bottom portion is connected to the chamber perimeter wall, the second retaining portion is configured inside the effusion space and unable to rotate relative to the chamber perimeter wall and/or the chamber bottom portion, and the second join portion is configured to the chamber perimeter wall;
wherein the second join portion enables joining to the first join portion, thereby completing assembly of the assembly member and the effusion chamber, wherein the BFS container is a liquid storage member, which is able to filed and clasped inside the cylindrical structure and the effusion space, moreover, under condition of not damaging the liquid storage member, the liquid storage member is unable to rotate about the axial direction of the liquid storage member as the rotation axis.

2. The medicine cup structure according to claim 1, wherein the assembly member is provided with an assembly member open end, the first join portion is configured to the assembly member open end, the first join portion is provided with a first threaded structure, the second join portion is provided with a second threaded structure, and the first threaded structure together with the second threaded structure is able to rotate relative to each other and be fixedly locked.

3. The medicine cup structure according to claim 2, wherein the first join portion is annular configured to an inner side of the cylindrical structure, the first join portion is provided with an internal threaded structure, the second join portion is disposed on an outer surface of the chamber perimeter wall, and is provided with an external threaded structure, and the internal threaded structure together with the external threaded structure is able to rotate relative to each other and be fixedly locked.

4. The medicine cup structure according to claim 3, wherein the first retaining portion further comprises a first elongated slot structure, and another end portion of the cylindrical structure is connected to the first elongated slot structure.

5. The medicine cup structure according to claim 4, wherein the second retaining portion comprises a second elongated slot structure.

6. The medicine cup structure according to claim 5, wherein the assembly member is further provided with an assembly member closed end corresponding to the assembly member open end; the extremity of the first elongated slot structure is the assembly member closed end, and the assembly member closed end is not connected to the cylindrical structure.

7. The medicine cup structure according to claim 6, wherein another end portion of the cylindrical structure assumes an arc shape.

8. The medicine cup structure according to claim 6, wherein the second retaining portion is formed by protruding from the chamber perimeter wall and/or the chamber bottom portion, or the second retaining portion is independently formed and then clasped to the chamber perimeter wall and/or the chamber bottom portion.

9. The medicine cup structure according to claim 8, wherein the assembly member further comprises a window structure, which is formed on a side wall of the cylindrical structure, the window structure is transparent or hollowed out.

10. The medicine cup structure according to claim 8, wherein the effusion chamber is additionally installed with an elastic rubber matrix that covers a surface of the second retaining portion.

11. The medicine cup structure according to claim 6, wherein another end portion of the cylindrical structure assumes an arc shape, the second retaining portion is formed by protruding from the chamber perimeter wall and/or the chamber bottom portion, or the second retaining portion is independently formed and then clasped to the chamber perimeter wall and/or the chamber bottom portion; the assembly member further comprises the window structure, which is formed on the side wall of the cylindrical structure, the window structure is transparent or hollowed out; moreover, the effusion chamber is additionally installed with the elastic rubber matrix that covers the surface of the second retaining portion.

12. The medicine cup structure according to claim 11, wherein a microporous membrane is also disposed on the chamber bottom portion, and the medicine cup structure assumes a liquid-tight state after completing assembly of the assembly member and the effusion chamber.

13. The medicine cup structure according to claim 1, wherein the stiffness of the assembly member and/or the effusion chamber is greater than the stiffness of the BFS container.

14. The medicine cup structure according to claim 1, wherein the liquid storage member is unable to rotate about the axial direction thereof as the rotation axis, which means the liquid storage member is unable to rotate as a whole body, or the angle that the liquid storage member is able to be twisted or rotated through is less than 30 degrees.

15. An assembly method for medicine cup structure, which is applicable for use to unseal a blow-fill-seal container (BFS container) filled with liquid medicine, wherein the BFS container is a liquid storage member, which comprises a second assembly portion and a liquid storage portion, wherein the liquid storage portion is connected to the second assembly portion, a first end portion and a second end portion are correspondingly configured on two ends of the liquid storage portion, and an extremity of the second end portion is provided with a protruding piece; the liquid storage portion is filled with the liquid medicine, and the protruding piece is connected to the second assembly portion; the assembly method of the medicine cup structure comprises the following steps of:
a) providing an assembly member step: which involves providing an assembly member, comprising a first retaining portion and a first join portion, the first join portion is connected to the first retaining portion, the first join portion is provided with a first threaded structure, the first retaining portion comprises a cylindrical structure, the first join portion is configured to the end portion of the cylindrical structure;
b) providing a liquid storage member step: which involves filling the liquid storage portion of the liquid storage member, and clasping the liquid storage portion inside the cylindrical structure, making it impossible for the liquid storage member to rotate about the axial direction of the liquid storage member as the rotation axis;
c) providing an effusion chamber step: which involves providing an effusion chamber, comprising an effusion space, a second retaining portion, and a second join portion, wherein the effusion space is structured from a chamber perimeter wall annular configured to a chamber bottom portion, the chamber bottom portion is connected to the chamber perimeter wall, the second retaining portion is configured inside the effusion space, and is unable to rotate relative to the chamber perimeter wall and/or the chamber bottom portion, and the second join portion is configured to the chamber perimeter wall, the second join portion is provided with a second threaded structure, and the first threaded structure together with the second threaded structure rotate relative to each other and are fixedly locked;
d) carrying out a clasping step: which involves rotating the second threaded structure of the second join portion relative to the first threaded structure of the first join portion, thereby enabling the assembly member and the effusion chamber to gradually approach and tighten together, until the second assembly portion is clasped inside the effusion space; moreover, under condition of not damaging the liquid storage member, the liquid storage member is unable to rotate around the rotation axis; and,
e) producing a liquid outlet opening step: which involves continued mutual tightening of the second join portion and the first join portion, further causing the second assembly portion to at least partially separate from the liquid storage portion, producing a liquid outlet opening, at which time, the liquid medicine in the liquid storage portion flows out from the liquid outlet opening and enters the effusion space.

16. The assembly method for medicine cup structure according to claim 15, wherein the liquid storage member comprises the first assembly portion, the two ends of the liquid storage portion respectively connect to the first assembly portion and the second assembly portion, the first assembly portion is provided with a first plate structure, the second assembly portion is provided with a second plate structure, the first retaining portion further comprises a first elongated slot structure, and the other end portion of the cylindrical structure is connected to the first elongated slot structure, wherein providing the assembly member step enables inserting the first plate structure of the first assembly portion of the liquid storage member into the first elongated slot structure, thereby restricting the first assembly portion to an interior of the first elongated slot structure and preventing rotation around the rotation axis.

17. The assembly method for medicine cup structure according to claim 16, wherein the second retaining portion comprises a second elongated slot structure, wherein carrying out the clasping step enables inserting the second plate structure of the second assembly portion into the second elongated slot structure, thereby restricting the second assembly portion to an interior of the second elongated slot structure, and preventing rotation around the rotation axis.

18. An atomization device, comprising the medicine cup structure as claimed in claim 1.
